(19) United States
(12) Patent Application Publication
(10) Pub. No.: US 2024/0082268 A1
(43) Pub. Date: Mar. 14, 2024

(54) METHODS AND COMPOSITIONS FOR CONTROLLING NEUROINFLAMMATION

(71) Applicant: Invenux, LLC, Fort Collins, CO (US)

(72) Inventor: Robert Swift, Fort Collins, CO (US)

(21) Appl. No.: 18/263,762

(22) PCT Filed: Jan. 31, 2022
(86) PCT No.: PCT/US2022/014583

§ 371 Date: Aug. 1, 2023

Related U.S. Application Data
(60) Provisional application No. 63/144,324, filed on Feb. 1, 2021.

(51) Int. Cl. A61K 31/00; A61K 36/185; A61P 25/28

(52) U.S. Cl. CPC A61K 31/658; A61K 36/185; A61P 25/28

(57) ABSTRACT

The disclosure provides methods and compositions comprising CBD for preventing, reducing and/treating neuroinflammation, and signs or symptoms or both of a disease associated with acute or chronic neuroinflammation in a subject in need thereof. A composition is provided for preventing or reducing microglia or astrocyte cell activation or both in a subject in need. Methods are provided for preventing or reducing signs or symptoms or both associated with excessive secretion of inflammatory signaling molecules in a subject in need thereof.

### CROSS REFERENCES TO RELATED APPLICATIONS

This application is being filed Jan. 31, 2022 as a PCT International Patent Application and claims the benefit of priority to U.S. Provisional Application No. 63/144,324, filed Feb. 1, 2021, which is incorporated by reference in its entirety.

### BACKGROUND OF THE DISCLOSURE

### Field of the Disclosure

The disclosure relates generally to use of therapeutically effective amounts of cannabinoids or functional equivalents, in particular cannabidiol (CBD), to prevent or reduce acute or chronic neuroinflammation in a subject in need thereof. Methods are provided for the treatment of subjects having acute or chronic neuroinflammation, or a neurodegenerative disease or disorder such as lysosomal storage disease, e.g.

Mucopolysaccharidosis III, Alzheimer's disease, stroke, dementia with Lewy bodies, Parkinson's disease, amyotrophic lateral sclerosis, stroke, central nervous system vasculitis, multiple sclerosis, spinal cord injury, traumatic brain injury, infection of the brain or central nervous system, and brain tumors.

### Description of the Related Art

Neuroinflammation is inflammation of nervous tissue in the peripheral or central nervous system. It may occur in response to a variety of triggers including disease, ischemia, trauma, infection, toxins, or auto-immune processes. Acute and chronic neuroinflammation can result from an inappropriate immune response that can lead to cell and tissue malfunction, damage and ultimately cell and tissue destruction. Activated microglia, activated astrocytes, activated macrophages, and activated mononuclear phagocytes and an increase in inflammatory signaling are often associated with neuroinflammation.

In recent years, it has been found the neuroinflammatory response is one of the major mechanisms causing neurodegeneration. That is, microglial or astrocyte cells or both present in the central nervous system may be activated by various exogenous and endogenous substances, and the activated microglial or astrocyte cells or both produce and release substances such as inflammatory signaling molecules, e.g. cytokines or chemokines or both, nitrogen monoxide, prostaglandin, peroxide and superoxide, (Gao et al., J Neurochem, 81, 1285-97, 2002; Nelson, P T. et al., Ann Med, 34, 491-500, 2002; Griffin, W. S. et al., J Neuroinflammation, 3, 5, 2006).

The production of such substances may provoke immune responses in the short run, but the excessive or continuous production of the substances induces that malfunction and the death of neighboring nerve cells to cause neurodegeneration. Also, since substances released by dying nerve cells induce the activity of microglial or astrocyte cells or both again, the neurodegeneration is caught in a continuous vicious circle. In fact, it was reported the activity of microglial or astrocyte cells or both is associated with various degenerative nerve diseases such as lysosomal storage diseases, Alzheimer's disease, stroke, dementia with Lewy bodies, Parkinson's disease, amyotrophic lateral sclerosis, stroke, multiple sclerosis, spinal cord injury, traumatic brain injury, infection of the brain or central nervous system, and brain tumors.

As such, considering the importance of the neuroinflammatory response in neurological disease, including neurodegenerative diseases, it is possible to prevent, reduce or treat neuroinflammation and neurodegenerative diseases that may develop therefrom by reducing the activity of microglial cells, astrocytes, and other immune cells, and thereby reduce neuroinflammation and the neurodegenerative process.

Prevention or reduction of the signs and symptoms of acute or chronic neuroinflammation may significantly prevent or slow or reduce progression and symptomatic manifestation of conditions associated with neuroinflammation. Individuals with lysosomal storage diseases, Alzheimer's disease, stroke, dementia with Lewy bodies, Parkinson's disease, amyotrophic lateral sclerosis, stroke, multiple sclerosis, spinal cord injury, traumatic brain injury, infection of the brain or central nervous system, and brain tumors present with neuroinflammation which contributes to the signs and symptoms of the disease.

Sanfilippo Syndrome or Mucopolysaccharidosis III (MPS III) is a rare pediatric autosomal recessive neurodegenerative lysosomal storage disease characterized by a relentless decline in cognition, progressive dementia, hyperactivity, aggressive behavior, loss of motor function, seizures, and death by late teens. Mutations in lysosomal enzymes cause accumulation of heparan sulfate (HS) in the lysosome of neuronal cells, causing neuroinflammation and neurodegeneration. It is believed reducing neuroinflammation in MPS III would provide clinical benefits. Improved methods and compositions for reducing pathophysiologic neuroinflammation are desirable.

Lysosomal storage diseases are caused by a loss of enzyme activity required for the metabolism of lipids, glycoproteins, and mucopolysaccharides found in the lysosome. The partially metabolized macromolecules build up in the lysosome causing lysosomal, cellular, and tissue dysfunction and neuroinflammation. Current therapies rely on enzyme replacement therapy (ERT) or human stem cell transplant (HSCT). Enzyme replacement therapy involves administering an intravenous solution containing the enzyme that is deficient or missing from the body and causing the disease. ERT does not treat the neurological manifestations of the disease. The enzymes used in ERT are too large to cross the blood-brain barrier (BBB) and the human stem cells do not sufficiently populate the central nervous system (CNS) compartment to treat neurological manifestations of the disease. Polgreen et al., “Pilot Study of the Safety and Effect of Adalimumab on Pain, Physical Function, and Musculoskeletal Disease in Mucopolysaccharidosis Types I and II.” Molecular Genetics and Metabolism Reports 10 (Jan. 15, 2017): 75-80. Tumor Necrosis Factor-alpha (TNF-alpha) is elevated in individuals with MPS I or II and associated with pain and physical dysfunction. A small pilot study with anti-TNF alpha monoclonal antibody Adalimumab suggests the antibody may improve physical function and possibly pain in children with MPS I or II. However, Adalimumab does not cross the BBB and alternative therapeutics are needed.

The hallmark of Alzheimer disease (AD) is the formation of amyloid plaques that are thought to be the cause of an irreversible progressive neurodegeneration that slowly deteriorates memory, thinking and behavior. The brain in AD shows a chronic neuroinflammatory response characterized by activated glial cells and increased expression of inflammatory signaling molecules. Experimental disease-modifying treatments for AD have targeted the formation and progression of plaques. These targeted therapies have not been successful (Atri, Alireza. “The Alzheimer's Disease Clinical Spectrum: Diagnosis and Management.” The Medical Clinics of North America 103, no. 2 (March 2019): 263-93.). Prevention or reduction of neuroinflammation in AD presents an opportunity prevent or reduce the signs and symptoms of AD through treatment with an anti-neuroinflammatory drug.

Chronic neuroinflammation is one of the hallmarks of Parkinson's disease (PD) pathophysiology. Post-mortem analyses of human PD patients and experimental animal studies reveal activation of microglial and astrocytes and an increases in pro-inflammatory mediators. Chronic release of pro-inflammatory cytokines and other inflammatory mediators by activated microglia and astrocytes leads to the exacerbation of dopaminergic neuron degeneration (Wang, Qinqin, Yingjun Liu, and Jiawei Zhou. “Neuroinflammation in Parkinson's Disease and Its Potential as Therapeutic Target.” Translational Neurodegeneration 4 (Oct. 12, 2015): 19-27). Prevention or reduction of neuroinflammation in PD presents an opportunity to prevent or reduce the signs and symptoms of PD through treatment with an anti-neuroinflammatory drug.

Multiple Sclerosis is a demyelinating neurological disease. It is inadequately treated by currently available therapies, and continue to produce progressive, severe, neurologic impairment in a large population of patients in the United States and worldwide. Multiple sclerosis is a chronic central nervous system disease characterized by immune-mediated demyelination of the white matter of the brain and spinal cord. Chronic and acute episodes associated with multiple sclerosis are treated by administering anti-inflammatory drugs like anti-TNF antibodies, however, anti-TNF antibodies to not cross the BBB and the diseases continues to progress with accumulating debilitation and death. Prevention or reduction of neuroinflammation in PD presents an opportunity to prevent or reduce the signs and symptoms of PD through treatment with an anti-neuroinflammatory drug.

Traumatic brain injury (TBI) is an injury caused by excessive force to the head that may cause external brain injury, brain dysfunction, or death. Traumatic brain injury is not just an acute injury, as it shares chronic symptoms with diseases such as Parkinson's and Alzheimer's. Neuroinflammation follows the initial impact and may persist for many years post-TBI. Neuroinflammation increases neural cell death by interfering with endogenous repair mechanisms and acts through immune cells, microglia, cytokines, chemokines, and other inflammatory molecules. The chronic neuroinflammation is manifested by extensive microglial and astrocyte activation and may be the most important cause of post-traumatic neurodegeneration in TBI (Lozano, Diego, Gabriel S Gonzales-Portillo, Sandra Acosta, Ike de la Pena, Naoki Tajiri, Yuji Kaneko, and Cesar V Borlongan. “Neuroinflammatory Responses to Traumatic Brain Injury: Etiology, Clinical Consequences, and Therapeutic Opportunities.” Neuropsychiatric Disease and Treatment 11 (Jan. 8, 2015): 97-106). Prevention or reduction of neuroinflammation post-TBI presents an opportunity to prevent or reduce the signs and symptoms of TBI through treatment with an anti-neuroinflammatory drug.

Amyotrophic lateral sclerosis (ALS) is a rapidly progressing neurodegenerative disease involving the degeneration of both upper and lower motor neurons in the motor cortex, brainstem and spinal cord (Brown, Robert H., and Ammar Al-Chalabi. “Amyotrophic Lateral Sclerosis.” The New England Journal of Medicine 377, no. 2 (Jul. 13, 2017): 162-72). Loss of motor neurons results in extensive paralysis commencing usually focally in the limbs or bulbar muscles. ALS is a universally fatal disease, typically due to respiratory failure between 2 and 5 years after diagnosis. Neuroinflammation is increasingly recognized as an important mediator of disease progression in patients with ALS, and is characterized by reactive CNS microglia and astrocytes, as well as infiltrating peripheral monocytes and lymphocytes, which can cause neuroinflammation with the release of proinflammatory mediators, neurotoxicity, neurodegeneration and disease progression accelerates. Prevention or reduction of neuroinflammation ALS presents an opportunity to prevent or reduce the signs and symptoms of ALS through treatment with an anti-neuroinflammatory drug.

Stroke is caused by a blockage in blood flow in the brain (ischemic stroke) or a rupture of a blood vessel in the brain (hemorrhagic stroke). This causes both an acute and chronic neuroinflammatory response in the brain. Current therapy for stroke is to reduce the ischemia or the bleeding, and thereby reduce the volume of necrotic tissue damage However, there is a well-known “penumbra effect” in ischemic stroke where the volume of damaged brain tissue is far greater than the initial region of necrotic tissue. The mechanisms of the penumbra effect may include activation of microglial, astrocytes and macrophages, oxidative stress, nitric oxide overproduction, release of inflammatory signaling, e.g. IL-1 alpha, IL-1 beta, IL-2, IL-4, IL-6, IL-8, Il-10, IL-17A, IL-23, INF-gamma, CCL2 (MCP-1), CXCL10 IP-10), CXCL1 (KC), MCP-1, CCL3 (MIP-1a), MIP-2, CCL5 (RANTES), caspase-1, caspase-3, TNF-alpha, TGF-alpha, and TGF-beta, expression of adhesion molecules, and production of matrix metalloproteinases. The penumbra is a target for neuroprotection, neurostabilization, and neurorepair where pharmacologic interventions are most likely to be effective. A challenge arises to reach this area with therapeutic agents due to reduced blood flow and the BBB. Castillo J. Evolving paradigms for neuroprotection: molecular identification of ischemic penumbra. Cerebrovasc Dis. 2006; 21(suppl 2):71-79. Prevention or reduction of neuroinflammation stroke presents an opportunity prevent or reduce the signs and symptoms of stroke through treatment with an anti-neuroinflammatory drug.

Improved treatments to reduce neuroinflammation are desirable, for example, a small lipophilic molecule that easily crosses the BBB and can rapidly diffuse into cells, tissues and the surrounding spaces may be able to overcome these challenges.

### SUMMARY OF THE DISCLOSURE

The present disclosure provides improved methods and compositions for preventing, or reducing neuroinflammation in a subject in need thereof.

The present disclosure provides a method of preventing or reducing acute or chronic neuroinflammation in a subject in need thereof, comprising administering a cannabinoid or functional equivalents, for example, CBD.

The present disclosure provides a method of preventing or reducing neurodegenerative disease or disorder in a subject in need thereof, comprising administering CBD.

The present disclosure provides a method of preventing or reducing acute or chronic neuroinflammation in a subject in need thereof, comprising administering to the subject a composition comprising a therapeutically effective amount of CBD and a pharmaceutically acceptable carrier.

The present disclosure provides a method of preventing or treating a disease or disorder associated with acute or chronic neuroinflammation in a subject in need thereof, comprising administering to the subject a composition comprising a therapeutically effective amount of CBD and a pharmaceutically acceptable carrier.

The present disclosure provides a method of preventing or treating neurodegeneration in a subject in need thereof, comprising administering to the subject a composition comprising a therapeutically effective amount of CBD and a pharmaceutically acceptable carrier.

In some embodiments, the composition may be administered via an oral, sub-lingual, buccal, sub-cutaneous, intramuscular, intraperitoneal, intracerebrovascular injection, intrarectal, e.g., enema or suppository, intrathecal, intravenous, intra-nasal, intra-lesion, topical, transdermal, transmucosal, or inhalation route. In some embodiments, the amount of CBD is effective to reduce severity or duration or both or delay onset of a disease or disorder associated with neuroinflammation in the subject.

In some embodiments, the composition for oral administration can be any appropriate orally acceptable dosage form. The orally acceptable dosage form may be selected from, but not limited to, capsules, tablets, films, lozenges, soft chews, gummies, emulsions and aqueous suspensions, dispersions, and solutions. In the case of tablets for oral use, carriers which are commonly used may include lactose and corn starch. Lubricating agents, such as magnesium stearate, may also be added. For oral administration in a capsule form, useful diluents may include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. A nasal aerosol or inhalation composition can be prepared according to techniques well-known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art. A cannabinoid composition of this disclosure can also be administered in the form of suppositories for rectal administration.

In some embodiments, the composition may administered as a sterile injectable composition, for example, a sterile injectable aqueous or oleaginous suspension, can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

In some embodiments, the disease or disorder associated with pathophysiologic neuroinflammation may be selected from the group consisting of a Lysosomal Storage Disease, Alzheimer's disease, Parkinson's disease, Amyotrophic Lateral Sclerosis, Lewy body dementia, Multiple Sclerosis, stroke, spinal cord injury, subacute combined degeneration of spinal cord, traumatic brain injury, CNS vasculitis, depression, schizophrenia, infection of the brain, infection of the central nervous system, and brain tumors. The neuroinflammation may be chronic neuroinflammation. The neuroinflammation may be acute neuroinflammation.

In some embodiments, the reduction of severity, duration, and/or delayed onset of neuroinflammation in the subject comprises reduction of microglia activation, reduction of astrocyte activation, and/or reduction of the secretion of inflammatory signaling molecules.

The reduction of the secretion of inflammatory signaling molecules may include a reduction in one or more, two or more, three or more biological markers (“biomarkers”) or signaling molecules, for example, selected from the group consisting of cluster of differentiation 68 (CD68), glial fibrillary acidic protein (GFAP), interleukin-1-alpha (IL-1-alpha), IL-1-beta, IL-2, IL-4, IL-6, IL-8, 11-10, IL-17A, IL-23, INF-gamma, CCL2 (MCP-1), CXCL10 IP-10), CXCL1 (KC), MCP-1, CCL3 (MIP-1α), MIP-2, CCL5 (RANTES), caspase-1, caspase-3, TNF-alpha, and TGF-beta.

The reduction of microglia activation may be evidenced by a reduction in CD68 expression on microglial cells.

The reduction of astrocyte activation may be evidenced by a reduction in GFAP expression on astrocyte cells.

The CBD may be a highly purified extract of Cannabis which comprises at least 95%, at least 97%, or at least 99% (w/w) CBD. The Cannabis may be a Cannabis sativa. In some embodiments, the CBD comprises no more than 2% THC, no more than 1% THC, or no more than 0.3% THC, for example, as determined by HPLC. The CBD may be a highly purified or synthetic CBD which comprises at least 98% (w/w) CBD.

In some embodiments, a composition is provided for use in a method of preventing or reducing acute or chronic neuroinflammation in a subject in need thereof, the method comprising administering to the subject a composition comprising a therapeutically effective amount of CBD and a pharmaceutically acceptable carrier.

In some embodiments, a composition is provided for use in treating or preventing a disease or disorder associated with acute or chronic neuroinflammation in a subject in need thereof, the composition comprising a therapeutically effective amount of CBD and a pharmaceutically acceptable carrier.

In some embodiments, a composition is provided for reducing severity or duration of symptoms of a disease or disorder associated with neuroinflammation in a subject in need thereof, the composition comprising a therapeutically effective amount of a CBD and a pharmaceutically acceptable carrier. In some embodiments, the symptoms of a disease or disorder associated with neuroinflammation may be selected from the group consisting of decline in cognition, progressive dementia, aggressive behavior, loss of motor function, seizures, paralysis, hemiplegia, confusion, dystonia, alexia, agnosia, and or impaired coordination.

In some embodiments, a composition is provided for use in manufacture of a medicament for treating or preventing a disease or disorder associated with acute or chronic neuroinflammation in a subject in need thereof, the composition comprising a therapeutically effective amount of CBD and a pharmaceutically acceptable carrier.

### BRIEF DESCRIPTION OF THE DRAWINGS

FIG. 1A shows a bar graph of the effects in the brain of 9-month old mice in a model of MPS IIIB using KO or Het mice after administering by oral gavage for 30 days with 100 mg/kg CBD in sesame oil or sesame oil (Veh), comparing the CD68% area immunoreactivity in brain slices obtained from MPS IIIB mice lacking NGLU enzyme activity (KO) given sesame oil vehicle [KO(Veh)], or CBD [KO(CBD)], and heterozygote control mice given vehicle [Het(Veh)], (sesame oil). The results show there was a 30% decrease in the immunoreactivity of CD68 in the MPS IIIB mice treated with CBD compared to the control mice. Brain slices were taken along the rostro caudal axis in a serial section. Quantification using the brain slices was performed using Image Pro Premiere software. As shown, CBD treatment significantly decreases microglia cell neuroinflammation and activation, as indicated by the reduction in CD68 associated with microglial cell activation.

FIG. 1B shows a bar graph of the effects in the brain of 9-month old mice in a model of MPS IIIB using KO or Het mice after administering by oral gavage for 30 days with 100 mg/kg CBD in sesame oil or sesame oil (Veh), comparing the GFAP % area immunoreactivity in brain slices obtained from MPS IIIB mice (KO) given sesame oil vehicle [KO(Veh)] or CBD [KO(CBD)] and heterozygote control mice given vehicle [Het(Veh)], sesame oil). The results show there was a 50% decrease in the immunoreactivity of GFAP in the MPS IIIB mice treated with CBD compared to the control mice. Brain slices were taken along the rostro caudal axis in a serial section. Quantification using the brain slices was performed using Image Pro Premiere software. As shown, CBD treatment significantly decreases astrocyte cell neuroinflammation and activation, as indicated by the reduction in GFAP associated with astrocyte cells activation.

FIG. 2 shows representative brain slices after immunostaining with CD68 antibodies (lower row) or GFAP antibodies (upper row) and taken along the rostro caudal axis in a serial section from MPS IIIB mice (KO). MPS IIB mice (KO) given sesame oil vehicle [KO(Veh)] or CBD [KO(CBD)] and heterozygote control mice given vehicle [Het(Veh)], sesame oil Serial brain slices were used to quantify the change in CD68 and GFAP immunostaining between MPS IIIB mice treated or not treated with CBD. That quantification is shown in FIG. 1A and FIG. 1B. As shown, CBD treatment significantly decreases both microglia and astrocyte cell neuroinflammation and activation, as indicated by the reduction in CD68 and GFAP associated with microglial and astrocyte cells activation, respectively.

### DETAILED DESCRIPTION

Cannabinoids are lipophilic small molecules that may readily cross the blood brain barrier (BBB) and bind to various receptors in the CNS.

CBD is a cannabinoid, devoid of psychoactive activity, derived from Cannabis sativa. CBD is a small molecule with a molecular weight of 314.5 g/mole. It is lipophilic with a log P value of 6.3. The logarithm of the partition coefficient between n-octanol and water is referred to as log P and is a way to characterize the lipophilicity of a molecule.

CBD can be extracted from Cannabis sativa or made synthetically.

The use, combination, composition or method according to the invention may be for preventing or reducing acute or chronic neuroinflammation and/signs or symptoms or both associated with acute or chronic neuroinflammation in a subject in need thereof.

The invention also pertains to compositions and methods for preventing or reducing or treating neurodegeneration and/signs or symptoms or both associated in a subject in need thereof.

The invention also pertains to compositions and methods of preventing or reducing microglia and astrocyte activation and/preventing signs or symptoms or both associated with excessive activation of microglia; or astrocyte or both and preventing or reducing the secretion of inflammatory signaling molecules like cytokines, particularly IL-1beta, IL-6, and TNF-alpha and preventing or reducing and treating signs or symptoms or both associated with excessive secretion of inflammatory signaling molecules.

In the context of the invention, the prophylactic treatment includes reducing the risk or occurrence of neuroinflammation or its symptoms, microglia and astrocyte activation and signs or symptoms or both and inflammatory signaling small molecule secretion and/or symptoms there or both of.

In the context of the present invention, the term “neuroinflammation” refers to an inflammation of the CNS. Herein, CNS refers to the brain and the spinal cord. In some instances, the present disclosure is directed to inflammation of the brain. In the context of the present disclosure, “secretion” (as in “secretion of inflammatory signaling molecules”) and “release” (as in “release of inflammatory signaling molecules”) are synonymous and used interchangeably.

The disclosure provides compositions comprising CBD and methods for preventing, reducing, or inhibiting activation of CNS microglial and astrocyte cells, and other immune cells, thereby reducing neuroinflammation.

Neuroinflammation is a response of the immune system of the CNS that is associated with many disorders, including lysosomal storage diseases, Alzheimer's disease. Dementia with Lewy bodies, Parkinson's disease, amyotrophic lateral sclerosis (AML), stroke, Multiple Sclerosis, spinal cord injury, traumatic brain injury, CNS vasculitis, infection of the brain or central nervous system, and brain tumors.

Microglia cells are thought to be the main important cell type involved in neuroinflammation. Microglia cells act as the first and main form of active immune defense in the CNS. Microglia cells have been found to be the primary source of brain cytokines and have been implicated in neuronal pathologies associated with chronic neuroinflammation. Microglia cells are the innate immune cells of the central nervous system, which act quickly on neuroinflammation. However, prolonged activation of microglia, as in chronic neuroinflammation, causes damage to brain tissue and to the BBB, causing neurodegenerative disorders. Microglial activation may be evidenced by an increase in CD68 or ionized calcium binding adaptor molecule 1 (Iba1) or both, changes in cell morphology, from highly ramified shape to flattening and shortening to an amoeba shape, and proliferation. Quantifying microglia morphology has been used in numerous studies as a key marker for microglial activation and function after CNS tissue injury, which can lead to release of damage associated molecular patterns (DAMPs), reactive oxygen species (ROS), cytokines, chemokines, and other proinflammatory mediators. Microglial change in microglial morphology from a highly ramified shape to an amoeba shape can be quantitatively assessed, according to Waller, Rachel, Lynne Baxter, Daniel J. Fillingham, Santiago Coelho, Jose M. Pozo, Meghdoot Mozumder, Alejandro F. Frangi, Paul G. Ince, Julie E. Simpson, and J. Robin Highley. “Iba-1−/CD68+ Microglia Are a Prominent Feature of Age-Associated Deep Subcortical White Matter Lesions.” PLoS ONE 14, no. 1 (Jan. 25, 2019): e0210888, using a metric that considers both shape and size of the individual cells, defined as the ‘morphology (M) metric’ for quantifying microglial cell morphology along the ‘ramified’ to ‘amoeboid’ spectrum as follows

$${M}_{i} = \{ \begin{matrix}{C}_{i} \frac{\text{?} - {A}_{0}}{{A}_{0}} , & if \text{?} \geq {A}_{0} , \\ 0 & if \text{?} < {A}_{0} ,\end{matrix}$$

$$\text{?} \text{indicates text missing or illegible when filed}$$

where i indexes the cells in the image. Aᵢ is the cell's total area and A₀ is the minimum area considered for the cells to be analyzed. cᵢ is the cell's roundness which is defined as cᵢ=4πAi/Pᵢ2, where Pᵢ is the cell's perimeter.

Activation, phagocytosis and cytokine release from microglia may be modulated by voltage-gated sodium channels. Without being bound by theory, CBD has micromolar affinity to the sodium channel Nav receptors and may bind to the Nav 1.6 receptors on microglia cells and prevent, modulate or reduce microglia activation, phagocytosis and cytokine release, thereby reducing neuroinflammation.

Astrocytes are the most abundant cells in the CNS. Under normal conditions astrocytes modulate synaptic activity, and provide nutrients and support needed for neuronal activity and survival. In the context of neuroinflammation, astrocytes control CNS infiltration by peripheral pro-inflammatory leukocytes, and regulate the activity of microglia, oligodendrocytes and cells of the adaptive immune system. Thus, therapeutic modulation of astrocyte activity is important for regulating astrocyte and microglial activation during neuroinflammation. Astrocyte activation is evidenced by an increase in glial fibrillary acid protein (GFAP), S100β, LDH1A1, proliferation, change in morphology somatic and dendritic hypertrophy, and processes elongation but not overall cellular volume change, and upregulation of structural and adhesion molecules, extracellular matrix (ECM) components and inflammatory chemokines and cytokines. These enhanced features along with cellular proliferation are called astrogliosis. A list of activated astrocyte markers can be found in Table 3 of Jurga, Agnieszka M., Martyna Paleczna, Justyna Kadluczka, and Katarzyna Z. Kuter. “Beyond the GFAP-Astrocyte Protein Markers in the Brain.” Biomolecules 11, no. 9 (Sep. 14, 2021): 1361. Activation and cytokine release of astrocytes may be modulated by sodium channels. Without being bound by theory, CBD has micromolar affinity to the sodium channel Nav receptors and may bind to the Nav 1.6 receptor on astrocytes and prevent, modulate or reduce astrocyte activation and cytokine release, thereby reducing neuroinflammation.

The present disclosure pertains to the use of therapeutically effective amounts of CBD for the manufacture of a composition or medicament for treating, reducing and/or preventing neuroinflammation and/or symptoms associated with neuroinflammation in a subject in need thereof, as well as for preventing, reducing, modulating or inhibiting microglia activation and/or reducing, modulating or inhibiting astrocyte activation and/or for treating, reducing and/or preventing symptoms associated with excessive activation of microglia and/or astrocyte activation and/or for treating, reducing and/or preventing symptoms associated with excessive secretion of inflammatory signaling molecules such as cytokines, chemokines and proteases in a subject in need thereof. Inflammatory signaling molecules may include one or more of, or two or more of IL-1-alpha, IL1-beta, IL-2, IL-4, IL-6, IL-8, Il-10, IL-17A, IL-23, INF-gamma, CCL2 (MCP-1), CXCL10 IP-10), CXCL1 (KC), CCL3 (MIP-1α), CCL5 (RANTES), caspase-1, caspase-3, TGF-alpha, TNF-alpha, and TGF-beta, preferably at least one of IL-1, IL-6 MCP-1, and TNF-alpha, most preferably at least IL-1, IL-6, and TNF-alpha. Excessive activation of microglia or astrocytes or both, or macrophages, or mononuclear phagocytes, or both or other immune cells causes damage to brain tissue and to the BBB causing neurodegenerative disorders.

CBD binds with uM affinity to the CB1 and CB2 endocannabinoid receptors and other receptors in the CNS, e.g. Nav 1.6 receptor.

The American Marijuana Organization lists an oral high dose of CBD for use in humans as 0.6 mg/kg/day.

In 2108, FDA approved Epidiolex (CBD in sesame oil) to treat patients with refractory epilepsy due to Lennox-Gastaut or Dravet syndrome. From the FDA approved label, the recommended starting dosage for Epidiolex is 2.5 mg/kg taken twice daily (5 mg/kg/day), which can be increased up to a maximum recommended maintenance dosage of 10 mg/kg twice daily (20 mg/kg/day). A dose that is 33.3 times the oral high dose listed by the American Marijuana Organization.

We define high dose CBD as greater than or equal to 5 mg/kg/day or about eight times the oral high dose listed by the American Marijuana Organization.

High dose Epidiolex (CBD), compared to the high dose of CBD listed by the American Marijuana Organization, may be necessary for Epidiolex to interact with low affinity CBD receptors in the CNS, e.g., CB2 and Nav 1.6. There is evidence Epidiolex exhibits its anti-seizure activity by blocking the Nav 1.6 receptor. CBD may bind to and block the voltage-gated sodium channel Nav 1.6 receptor in the CNS with an IC₅₀ from 1-6 uM.

There is evidence that activation of the Nav 1.6 receptor on microglia and astrocytes can activate these cells and cause the release of proinflammatory mediators from these cells. Therefore, in some embodiments the present disclosure provides methods and compositions for providing high dose CBD, greater than or equal to 5 mg/kg/day, to achieve an anti-neuroinflammatory activity by blocking Nav 1.6 receptor and preventing or reducing the activation of microglia or astrocytes or both in the CNS.

In some embodiment, the high dose CBD binds to and blocks signaling or activates signaling of the CB2 receptor to achieve an anti-neuroinflammatory activity on microglia or astrocytes or both.

In some embodiment, the high dose CBD binds to and blocks signaling or activates signaling of the of other receptor in the CNS for which CBD has a low affinity, in the range of 0.2 to 20 uM to achieve an anti-neuroinflammatory activity on microglia or astrocytes or both.

In some embodiments, the high dose of CBD may be greater than 20 mg/kg/day, greater than about 25 mg/kg/day, greater than about 30 mg/kg/day, greater than about 40 mg/kg/day, greater than 50 mg/kg/day and less than or equal to 100 mg/kg/day.

In some embodiments, the dose may be in a range of 10-100, 10-90, 10-80, 10-70, 10-60, 10-50, 10-40, or 10-30 mg/kg/day of CBD. In some embodiments, the dose may be in a range of 20-100, 20-90, 20-80, 20-70, 20-60, 20-50, 20-40, or 20-30 mg/kg/day of CBD. In some embodiments, the dose may be in a range of 25-100, 25-90, 25-80, 25-70, 25-60, 25-50, 25-40, or 25-30 mg/kg/day of CBD. In some embodiments, the dose may be in a range of 30-100, 30-90, 30-80, 30-70, 30-60, 30-50, 30-40, or 30-35 mg/kg/day of CBD. In some embodiments, the dose may be in a range of 40-100, 40-90, 40-80, 40-70, 40-60, 40-50, or 40-45 mg/kg/day of CBD.

In some embodiments, the maximum plasma concentration (Cₘₐₓ) of high dose CBD may be greater than 30 ng/ml, greater than 95 ng/ml, greater than 280 ng/ml and less than or equal to 850 ng/ml.

In some embodiments, the Cₘₐₓ may be in the range of 280-850 ng/ml.

In some embodiments, the Cₘₐₓ may be in the range of 50-280 ng/ml.

CBD binds to a hydrophobic pocket present between the voltage-gate sodium channel Nav 1.6. subunits and blocks the ingress of sodium (Sait, Lily Goodyer, Altin Sula, Mohammad-Reza Ghovanloo, David Hollingworth, Peter C Ruben, and BA Wallace. “Cannabidiol Interactions with Voltage-Gated Sodium Channels.” Edited by Leon D Islas, Olga Boudker, and Leon D Islas. ELife 9 (Oct. 22, 2020): e58593).

Blocking the Nav 1.6 receptor can prevent or reduce activation of microglial or astrocyte cells or both.

Prevention or reduction in activation of microglial or astrocyte cells or both can reduce their secretion of proinflammatory mediators.

Prevention or reduction in secretion of proinflammatory mediators in the CNS can prevent or reduce neuroinflammation.

Changes from baseline in the activation state of microglial and astrocytes can be measured by magnetic resonance spectroscopy and other appropriate imaging technology, e.g., magnetic resonance imaging (MRI) and positron emission tomography (PET).

When microglia are activated from their resting state, they can express high levels of the 18-kDa translocator protein (TSPO), which can be measured in vivo in the brain with the PET) radiotracer [¹¹C]PBR28.

When astrocytes are activated from their resting state they bind various PET radiotracers, e.g. imidazoline2 binding sites (I2BS) (Liu, Yu, Han Jiang, Xiyi Qin, Mei Tian, and Hong Zhang. “PET Imaging of Reactive Astrocytes in Neurological Disorders.” European Journal of Nuclear Medicine and Molecular Imaging, Dec. 7, 2021).

A change in neuroinflammation from baseline can be measured by magnetic resonance spectroscopy imaging (MRSI) and other appropriate imaging technology, e.g., magnetic resonance imaging and positron emission tomography (PET).

A change in neuroinflammation from baseline can be measured by whole-brain MRSI scan, which provides metabolite concentrations in 4,000 separate voxels, giving whole-brain coverage.

A change in neuroinflammation from baseline can be measured by the change in concentration of proinflammatory mediators found in plasma or cerebral spinal fluid CSF) or both, e.g. IL-1beta, IL-6, and TNF-alpha or GFAP.

In some embodiments, a method is provided of preventing, inhibiting, and/or modulating microglia activation, comprising the administration to a human subject in need thereof a therapeutic dose of one or more cannabinoids or functional equivalent, in particular of CBD therapeutic dose of one or more cannabinoids or functional equivalent, in particular of CBD.

In some embodiments, a method of inhibiting the release of inflammatory signaling molecules from activated microglial cells is provided, comprising administering to a human subject in need an appropriate therapeutic dose of one or more cannabinoids or functional equivalent, in particular of CBD.

In some embodiments, a method of preventing inhibiting or modulating astrocyte activation is provided, comprising the administration to a human in need thereof an appropriate therapeutic dose of one or more cannabinoids or functional equivalent, in particular of CBD.

In some embodiments, a method of inhibiting the release of inflammatory signaling molecules from activated astrocyte cells is provided, comprising the administration to a human in need an appropriate therapeutic dose of one or more cannabinoids or functional equivalent, in particular of CBD.

In some embodiments, a method of treating a disease or disorder associated with neuroinflammation is provided, comprising administering to a subject in need thereof a composition comprising a therapeutically effective amount of CBD and a pharmaceutically acceptable carrier. The neuroinflammation may be CNS neuroinflammation. The neuroinflammation may be peripheral neuroinflammation. In some embodiments, the neuroinflammation may be CNS and peripheral neuroinflammation. In some embodiments, the disease or disorder associated with neuroinflammation is selected from the group consisting of lysosomal storage diseases, Alzheimer's disease, stroke, dementia with Lewy bodies, Parkinson's disease, amyotrophic lateral sclerosis, stroke, CNS vasculitis, Multiple Sclerosis, spinal cord injury, traumatic brain injury, infection of the brain or central nervous system, and brain tumors. In some embodiments, a method if provided for neuroprotection and/or nerve regeneration in a subject suffering from a disease or disorder associated with excessive production of inflammatory signaling molecules in the brain, comprising the administration to a human in need thereof an appropriate therapeutic dose of one or more cannabinoids or functional equivalent, in particular of CBD.

Yet other embodiments and aspects of the invention will be apparent according to the description provided below.

As used herein, the term “treating”, “treat” or “treatment” refers to. (i) preventing a disease, disorder or condition from occurring in an animal or human that may be predisposed to the disease, disorder and/or condition but has not yet been diagnosed as having it; (ii) inhibiting the disease, disorder or condition, i.e., arresting its development; or (iii) relieving the disease, disorder or condition, i.e., causing regression of the disease, disorder and/or condition (iv) slowing progression of the disease, disorder or condition or (v) reducing the signs or symptoms or both of the disease, disorder or condition. For example, as indicated above, treating does not necessarily indicate a reversal or cessation of the disease, disorder or condition afflicting the subject being treated, but could encompass the lessening or reduction in the deleterious signs, symptoms, and/or rate in the progression of the disease, disorder, or condition being treated as compared to that which would occur in the absence of treatment. As understood, a change in the sign, symptom or rate may be assessed at the level of the subject (e.g., the function or condition of the subject is assessed), or at a tissue or cellular level (e.g., the production and/or release of pro-inflammatory substances or inflammation signaling molecules from activated microglial cells is lessened or reduced). For example, with respect to MPS III or multiple sclerosis treatment may be measured by quantitatively or qualitatively to determine the presence/absence of the disease, or its progression or regression using, for example, symptoms associated with the disease or clinical indications associated with the pathology, such as changes in movement or range of motion or both, or changes in neurocognitive test from a baseline prior to treatment as measured over time, i.e. measuring disease progression.

As used herein, the term “treat” or “treatment” of neuroinflammation includes prophylaxis and typically involves controlling neuroinflammation, preferably to the extent that (pathological or detriment) neuroinflammation is measurably contained, confined or reduced. “Detrimental” or“pathological” neuroinflammation may be acute or chronic neuroinflammation. In the context of the invention, treating or reducing neuroinflammation or both includes reducing the intensity of (detriment) neuroinflammation or reducing the duration of (detriment) the neuroinflammation or both and clinical indications associated with the neuroinflammation pathology.

Surprisingly, it has been found that CBD reduces the activation of microglia and astrocytes in MPS IIIB mice. MPS IIIB is lysosomal storage disease. We dosed MPS IIIB mice for 30 days at 100 mg/kg/day with CBD dissolved in sesame oil. The 9-month old MPS IIIB mice are very sick and are generally euthanized at 9 months, so any reduction in the activation of microglial and astrocytes by CBD is even more surprising as it is very difficult to effect change in such a sick animal with such progressive disease.

A human patient may be treated with CBD prophylactically, if at risk for neuroinflammation and treated with CBD if they are suspected of having neuroinflammation, or if confirmed by testing they have a neuroinflammation or they have a disease suspected or known to cause neuroinflammation.

Based on the present in vivo mouse data, one dosage range for preventing or treating neuroinflammation in a human subject may be about 10 to about 30 mg/kg/day. In some embodiments, the 10-30 mg/kg/day may be divided into two doses given approximately 12 hours apart (q12) or three doses given approximately 8 hours apart (q8). It is possible in some patients a lower dose or different dosing schedule will be effective. We expect the range of effective dosing and schedule to prevent or treat neuroinflammation in humans is 10-50 mg/kg/day given QD q12 or q8. This dose is similar to the dose of Epidiolex to reduce seizures in rare forms of pediatric epilepsy. CBD does not appear to exert its anti-seizure effects through interaction with cannabinoid receptors, e.g., CB1, but it is hypothesized the CBD anti-seizure effects are mediated through a class of voltage-gated sodium channels (Nav1.1-9). We hypothesize the anti-neuroinflammatory evidenced by CBD may be mediated through Nav1.6, which is found on microglia and astrocytes and known to modulate their activation. The IC₅₀ of CBD for the Nav′.6 receptor is 1-6 uM. Activation of the Nav1.6 receptor increases microglia phagocytosis and inflammatory signaling molecule release. Activation of the Nav1.6 receptor increases astrocyte inflammatory signaling molecule release. This is consistent with the high dose of CBD necessary to affect neuroinflammation and the micromolar affinity of CBD for the Nav1.6 receptor.

In one embodiment, the composition according to the invention is for treating and/or preventing neuroinflammation, preferably for treating neuroinflammation, particularly reducing the duration and/or extent of neuroinflammation.

In one embodiment, the neuroinflammation is neuroinflammation of the central nervous system. Also, in the context of the invention, microglia activation and/or inflammatory cytokine secretion is typically reduced in the central nervous system. Excessive activation of microglia causes damage to brain tissue and to the BBB, causing neurodegenerative disorders. Also, in the context of the invention, astrocyte activation and/or inflammatory cytokine secretion is typically reduced in the central nervous system. Excessive activation of astrocytes causes damage to brain tissue and to the BBB, causing neurodegenerative disorders

In some embodiments, the neuroinflammation may be caused by toxins or toxic metabolites, autoimmunity, aging, infection (e.g. bacterial or viral), traumatic brain injury, stroke, for example, stroke-associated or stroke-induced neuroinflammation. In some embodiments, the targeted subject suffers from stroke, has suffered from stroke, is at increased risk of stroke or is at increased risk of recurrent stroke. In some embodiments, the present use, method, combination or composition for use is for preventing and/or reducing the risk of recurrent stroke or a second or further occurrence of stroke. In some embodiments, the targeted subject has suffered from stroke.

In some embodiments, the use, combination, composition or method according to the invention is for therapeutically reducing the secretion of inflammatory signaling molecule like cytokines or chemokines or both r for treating, reducing or preventing symptoms associated therewith. The reduction or decrease in inflammatory cytokine secretion may take the form of a reduced amount (extent) of (excessive) expressed inflammatory cytokine and/or a reduced duration of (excessive) inflammatory cytokine secretion. Such reduction in inflammatory cytokine secretion typically occurs in the central nervous system. In some embodiments, the inflammatory signaling molecule may include one of the following: IL-1 alpha, IL1 beta, IL-2, IL-4, IL-6, IL-8, Il-10, IL-17A, IL-23, INF-gamma, CCL2 (MCP-1), CXCL10 IP-10), CXCL1 (KC), CCL3 (MIP-1α), CCL5 (RANTES), caspase-1, caspase-3, TNF-alpha, TGF-alpha, and TGF-beta. For example, at least one of IL-1, IL-6 MCP-1, and TNF-alpha, or at least IL-1, IL-6, and TNF-alpha. Inflammatory cytokines such as IL-1, IL-6 and TNF-alpha are typically expressed by activated microglia during neuroinflammation, and lead to a prolonged state of increased oxidative stress.

In some embodiments a subject in need of treatment may be given a loading dose of CBD of 40 mg/kg/day for up to 7 days, and then their dose is reduced to 5-30 mg/kg/day. In some embodiments, a subject in need of treatment may be given a dose of CBD of 5-30 mg/kg/day without a loading dose.

In some embodiments, CBD is formulated as an oral solution or capsule in any suitable edible oil. CBD may be formulated as an oral solution or capsule in soybean oil. CBD may be formulated as an oral solution or capsule in niger oil.

In some embodiments, optionally a flavorant, sweetener, and or coloring agent may be added to the CBD oral solution.

In some embodiments, the CBD may be formulated as an oral solution further comprising a flavorant and a sweetener.

In some embodiments, the CBD may be formulated as a liposome. The oral bioavailability of CBD in sesame oil given orally is about 10-20%. Oral bioavailability is measured by dividing the amount of CBD in plasma after administered orally by the amount of CBD in plasma after administered intravenously. By definition, when a medication is administered intravenously, its bioavailability is 100% Millar, Sophie A., Nicole L. Stone, Andrew S. Yates, and Saoirse E. O'Sullivan. “A Systematic Review on the Pharmacokinetics of Cannabidiol in Humans.” Frontiers in Pharmacology 9 (2018).

Formulation in a liposome is likely to increase bioavailability thereby resulting in a lower dose of CBD per day or allow an increase in CBD dose with fewer side-effects.

In one embodiment, CBD is formulated with nanoparticles. Formulation with nanoparticles is likely to increase bioavailability thereby resulting in a lower dose of CBD per day or allow an increase in CBD dose with fewer side-effects.

In one embodiment, CBD is formulated as an emulsion. Formulation in as an emulsion is likely to increase bioavailability thereby resulting in a lower dose of CBD per day or allow an increase in CBD dose with fewer side-effects.

In one embodiment, CBD is formulated for transdermal delivery. Formulation for transdermal delivery is likely to increase bioavailability thereby resulting in a lower dose of CBD per day or allow an increase in CBD dose with fewer side-effects. In particular, transdermal delivery will bypass first-pass liver metabolism, increase bioavailability, and reduce GI side-effects.

In one embodiment, CBD is formulated for rectal delivery, e.g. enemas or suppositories. Formulation for rectal delivery is likely to increase bioavailability thereby resulting in a lower dose of CBD per day or allow an increase in CBD dose with fewer side-effects. In particular, rectal delivery will bypass first-pass liver metabolism, increase bioavailability, and reduce GI side-effects.

### Definitions

The term “cannabinoid” refers to a class of diverse chemical compounds found in the Cannabis plant. The word “Cannabis” refers to all products derived from the plant Cannabis sativa. The Cannabis plant contains about 540 chemical substances. Besides CBD, more than 100 other cannabinoids have been identified. Synthetic cannabinoids encompass a variety of distinct chemical classes: the classical cannabinoids structurally related to CBD, the nonclassical cannabinoids including the aminoalkylindoles, 1,5-diarylpyrazoles, quinolones, and arylsulphonamides, as well as eicosanoids related to the endocannabinoids.

The term “neuroinflammation” or “neuroinflammatory diseases, disorders or conditions,” as used herein, includes diseases, disorders and conditions that are associated with the central and peripheral nervous systems, It may occur in response to a variety of triggers including disease, trauma, infection, toxins, or auto-immune processes, and inflammation that causes destruction of healthy neuronal and/or cerebral tissue. Inflammation is a complex biological response that is basic to how the body addresses injury and infection to eliminate the initial cause of cell injury and repair tissues. Acute and chronic inflammation often results from an inappropriate immune response that can lead to tissue damage and ultimately tissue destruction. Inflammation in the nervous system or “neuroinflammation,” especially when prolonged, can be particularly harmful. While inflammation per se may not cause disease, it contributes importantly to the process of disease in both the peripheral and central nervous systems. Treatment of neuroinflammation may significantly impact the progression and symptomatic manifestation of those conditions associated with neuroinflammation.

The term pathophysiologic neuroinflammation refers to abnormal or disease or syndrome associated neuroinflammation.

Within the context of the invention, “symptoms associated with neuroinflammation” may be referred to as symptoms of neuroinflammation, and it is well within the skilled person's ambit to appreciate which are symptoms of neuroinflammation.

As used herein, the term “modulates” refers to an increase or decrease as compared to a control, for example, CBD may decrease an activity, expression level, symptom, condition, or progression of a disease or disorder associated with neuroinflammation as compared to a control or that which would occur in the absence of CBD, for example, the ability to increase or decrease the amount of pro-inflammatory substances or inflammation signaling molecules produced within or released from a cell, such as a microglial cell and/or astrocyte.

As used herein, the term “inhibiting” or “inhibits” refers to the ability to significantly reduce or decrease a level, an amount, an activity, the severity of a disease, disorder, condition, or symptom and the like, e.g. of inflammatory signaling molecule, anti-inflammatory substance, mRNA, or protein, or biomarker, or activity, for example, as compared to a control. In some embodiments, “inhibits” refers to a decrease of at least 10%, or at least 20% of a level, an amount, an activity, the severity, frequency, or duration of a disease, disorder, condition, or symptom and the like, e.g., of anti-inflammatory signaling molecule, anti-inflammatory substance, mRNA, or protein, or biomarker, or activity, for example, as compared to a control.

As used herein, the term “neurotoxin” or “neurotoxicant” includes a substance that injures, damages, or kills a neuron.

As used herein, the term “pharmaceutically acceptable carrier” refers to any carrier, diluent, excipient, disintegrant, wetting agent, buffering agent, binder, suspending agent, lubricating agent, adjuvant, vehicle, delivery system, emulsifier, disintegrant, absorbent, preservative, glidant, polishing agent, surfactant, lubricant, opaquant, direct compression excipient, colorant, flavorant, sweetening agent, antiadherent, as known in the art that would be suitable for use in a pharmaceutical composition.

As used herein, the term “disintegrant” is intended to mean a compound used in solid dosage forms to promote the disruption of the solid mass into smaller particles which are more readily dispersed or dissolved. Such disintegrants include, by way of example and without limitation, starches such as corn starch, potato starch, pregelatinized and modified starches thereof, sweeteners, clays, such as bentonite, microcrystalline cellulose (e.g., Avicel), carboxymethylcellulose calcium, cellulose polyacrilin potassium (e.g., Amberlite), alginates, sodium starch glycolate, gums such as agar, guar, locust bean, karaya, pectin, tragacanth and other materials known to one of ordinary skill in the art.

As used herein, the term “colorant” is intended to mean a compound used to impart color to solid (e.g., tablets) or liquid pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and ferric oxide, red, other F.D. & C. dyes and natural coloring agents such as grape skin extract, beet red powder, beta-carotene, annato, carmine, turmeric, paprika, and other materials known to one of ordinary skill in the art. The amount of coloring agent used varies as desired.

As used herein, the term “flavorant” is intended to mean a compound used to impart a pleasant flavor and often odor to a pharmaceutical preparation. Exemplary flavoring agents or flavorants include synthetic flavor oils and flavoring aromatics and/or natural oils, extracts from plants, leaves, flowers, fruits and so forth and combinations thereof. These may also include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Other useful flavors include vanilla, citrus oil, including lemon, orange, grape, lime and grapefruit, and fruit essences, including apple, pear, peach, strawberry, raspberry, cherry, plum, pineapple, apricot and so forth. Flavors which have been found to be particularly useful include commercially available orange, grape, cherry and bubble gum flavors and mixtures thereof. The amount of flavoring may depend on a number of factors, including the organoleptic effect desired. Flavors will be present in any amount as desired by those of ordinary skill in the art. For example, flavors may include cherry flavors and citrus flavors such as orange or pomegranate.

As used herein, the term “sweetening agent” is intended to mean a compound used to impart sweetness to a preparation. Such compounds include, by way of example and without limitation, aspartame, dextrose, glycerin, mannitol, saccharin sodium, sorbitol and sucrose and other materials known to one of ordinary skill in the art.

As used herein, the term “antiadherent” is intended to mean an agent that prevents the sticking of tablet formulation ingredients to punches and dies in a tableting machine during production. Such compounds include, by way of example and without limitation, magnesium stearate, talc, calcium stearate, glyceryl behenate, PEG, hydrogenated vegetable oil, mineral oil, stearic acid and other materials known to one of ordinary skill in the art.

As used herein, the term “binder” is intended to mean a substance used to cause adhesion of powder particles in tablet granulations. Such compounds include, by way of example and without limitation, Copovidone (Kollidon VA-64) NF, alginic acid, carboxymethylcellulose sodium, poly(vinylpyrrolidone), compressible sugar (e.g., NuTab™), ethylcellulose, gelatin, liquid glucose, methylcellulose, povidone and pregelatinized starch and other materials known to one of ordinary skill in the art.

When needed, a binder may also be included in the present compositions. Exemplary binders include acacia, tragacanth, gelatin, starch, cellulose materials such as methyl cellulose and sodium carboxy methyl cellulose, alginic acids and salts thereof, polyethylene glycol, guar gum, polysaccharide, bentonites, sugars, invert sugars, poloxamers (PLURONIC F68, PLURONIC F127), collagen, albumin, gelatin, cellulosics in nonaqueous solvents, combinations thereof and others known to those of ordinary skill. Other binders include, for example, polypropylene glycol, polyoxyethylene-polypropylene copolymer, polyethylene ester, polyethylene sorbitan ester, polyethylene oxide, combinations thereof and other materials known to one of ordinary skill in the art.

As used herein, the term “diluent” or “filler” is intended to mean an inert substance used as filler to create the desired bulk, flow properties, and compression characteristics in the preparation of tablets, capsules, caplets, lozenge, troche, effervescent tablet, gummies, disintegrable tablets, and liquids. Such compounds include, by way of example and without limitation, dibasic calcium phosphate, kaolin, lactose, sucrose, mannitol, microcrystalline cellulose (e.g., Microcrystalline Cellulose PH101 NF, and Microcrystalline Cellulose PH200 NF), powdered cellulose, precipitated calcium carbonate, sorbitol, and starch, vegetable oils, and other materials known to one of ordinary skill in the art.

As used herein, the term “direct compression excipient” is intended to mean a compound used in direct compression tablet formulations. Such compounds include, by way of example and without limitation, dibasic calcium phosphate (e.g., Ditab) and other materials known to one of ordinary skill in the art.

As used herein, the term “glidant” is intended to mean agents used in tablet and capsule formulations to promote the flowability of a granulation. Such compounds include, by way of example and without limitation, colloidal silica, colloidal silicon dioxide NF, cornstarch, talc, calcium silicate, magnesium silicate, colloidal silicon, silicon hydrogel and other materials known to one of ordinary skill in the art. In some embodiments, the pharmaceutical composition further comprises a granulation solvent, e.g., purified water USP.

As used herein, the term “lubricant” is intended to mean substances used in tablet formulations to reduce friction during tablet compression. Such compounds include, by way of example and without limitation, magnesium stearate NF, calcium stearate, magnesium stearate, mineral oil, stearic acid, and zinc stearate and other materials known to one of ordinary skill in the art.

As used herein, the term “opaquant” is intended to mean a compound used to render a capsule or a tablet coating opaque. May be used alone or in combination with a colorant. Such compounds include, by way of example and without limitation, titanium dioxide and other materials known to one of ordinary skill in the art.

As used herein, the term “polishing agent” is intended to mean a compound used to impart an attractive sheen to coated tablets. Such compounds include, by way of example and without limitation, carnauba wax, and white wax and other materials known to one of ordinary skill in the art.

As used herein, the terms “pharmaceutically effective” or “therapeutically effective” shall mean an amount of CBD that is sufficient to show a meaningful patient benefit, i.e., treatment, prevention, amelioration, or a decrease in the frequency, duration, and/or severity, of the condition or symptom being treated.

The terms “patient”, “subject” and “recipient” are used interchangeably herein. The subject may be a human subject. The subject may be a non-human animal such as a monkey, a dog, a cat, a rabbit, a guinea pig, a rat, a mouse, cattle, a sheep, a pig, a goat, etc., birds, or fish.

CBD may be administered in the form of a pharmaceutical composition with a pharmaceutically acceptable carrier. The term “effective amount” means a dosage sufficient to provide treatment for the disease, disorder or condition being treated. For example, an effective amount of CBD can be an amount that decreases the release of pro-inflammatory substances or inflammation signaling molecules from activated microglial cells or astrocytes compared to that which would occur in the absence of CBD and may treat, prevent, ameliorate, or decrease a frequency, duration, and/or severity, of the condition or symptom associated with or resulting from the release of pro-inflammatory substances or inflammation signaling molecules such as neuroinflammation.

As used herein, the term “inflammatory signaling molecules” refers to substances produced within and/or released from a microglial cell, astrocyte, macrophage, or mononuclear phagocyte, preferably an activated microglial or astrocyte cell, mononuclear phagocyte or macrophage, that decrease inflammation. Inflammatory signaling molecules include but are not limited to cytokines, chemokines, and proteases such as IL-1-alpha, IL-1-beta, IL-2, IL-4, IL-6, IL-8, Il-10, IL-17A, IL-23, INF-gamma, CCL2 (MCP-1), CXCL10 IP-10), CXCL1 (KC), MCP-1, CCL3 (MIP-1α), MIP-2, CCL5 (RANTES), caspase-1, caspase-3, TNF-alpha, and TGF-beta. As used herein, the term “pro-inflammatory substances” or “inflammation signaling molecules” refers to substances produced within and/or released from and/or induced by a microglial cell, astrocytes, mononuclear phagocyte, or macrophage, preferably an activated microglial cell, astrocyte, mononuclear phagocyte, or macrophage that promote inflammation.

Compositions

Compositions are provided comprising a therapeutically effective amount of CBD and a pharmaceutically acceptable carrier. Cannabidiol (CAS RN 13956-29-1) may be obtained by any known method or may be purchased commercially. In some embodiments, CBD may be isolated from plant material by supercritical CO₂ extraction. To extract CBD from a Cannabis sativa or a hemp plant, ground Cannabis or hemp plant material comprised substantially of inflorescences and leaves may be charged into extracting vessels and supercritical CO₂ is passed through Cannabis or hemp in an extractor, for example, according to U.S. Pat. No. 10,870,632. Alternatively, CBD may be purchased commercially from a number of suppliers, for example Bluebird Botanicals of Louisville, Colorado.

In some embodiments, the CBD is greater than 75% pure analytically, for example by HPLC, HPLC-MS, HPLC-MS/MS, GC, or the like. In some embodiments, the CBD may be greater than 80%, 85%, 90%, 95%, 97%, 98%, 99%, 99.5%, or more purity. In some embodiments, the CBD has no more than 20%, 10%, 5%, 1%, or 0.3% of an impurity. In some embodiments, the CBD comprises no more than 0.3% THC impurity.

In some embodiments, the composition according to the invention may be used as a pharmaceutical product comprising one or more pharmaceutically acceptable carrier materials. Such product may contain the daily dosages as defined below in one or more dosage units. The dosage unit may be in a liquid form or in a solid form, wherein in the latter case the daily dosage may be provided by one or more solid dosage units, e.g. in one or more capsules or tablets. The pharmaceutical product, preferably for enteral application, may be a solid or liquid galenical formulation. Examples of solid galenical formulations are tablets, capsules (e.g. hard or soft shell gelatine capsules), caplets, pills, lozenge, troche, effervescent tablet, gummies, disintegrable tablets, sachets, powders, granules and the like which contain the active ingredients together with conventional galenical carriers. The composition may be in the form of a liquid. Any conventional carrier material can be utilized. The carrier material can be organic or inorganic inert carrier material suitable for oral administration. Suitable carriers include vegetable oils, gelatine, gum Arabic, lactose, starch, magnesium stearate, talc, and the like. Additionally, additives such as flavoring agents, sweeteners, preservatives, stabilizers, emulsifying agents, buffers and the like may be added in accordance with accepted practices of pharmaceutical compounding.

Administration

The pharmaceutical composition may be administered in an amount effective to reduce neuroinflammation or reduce symptoms of neuro-degenerative diseases in a subject. A preferred dose of the pharmaceutical composition according to the present invention may vary depending on the condition and weight of a subject, the severity of a disease, the shape of a drug, a route of administration, and an administration period, but may be properly chosen by those skilled in the related art.

The pharmaceutical composition may be administered by an oral, sub-lingual, buccal, sub-cutaneous, intramuscular, intraperitoneal, intracerebrovascular injection, intrarectal, intrathecal, intravenous, intra-nasal, intra-lesion, topical, transdermal, transmucosal, rectal, e.g. enema or suppository, or inhalation route, or at a daily dose appropriate for the route of administration. In some embodiments, the composition is administered orally.

The daily dose may be administered QD, BID or TID, e.g., for chronic treatment.

In some embodiments, a subject in need of treatment may be given a loading dose of CBD of 40 mg/kg/day for up to 7 days, and then a reduced dose of 10-30 mg/kg/day.

The composition may be administered at a higher daily dose between from about 40 to about 100 mg/kg/day, about 75 to about 150 mg/kg/day, or about 80 to about 120 mg/kg/day CBD, for example, for acute treatment, lasting less than seven days, for example, in an ischemic stroke.

### EXAMPLES

### Example 1. Effect of CBD Treatment on Cellular Neuroinflammation in Mouse Model of MPS IIIB

In this example, a mouse model of the lysosomal storage disease MPS IIIB was employed to assess the effects in the brain of an oral CBD composition compared to vehicle control on biomarkers of neuro-inflammation. An oral administration of 98% plus purity CBD in sesame oil vehicle or sesame oil vehicle control (Veh) was administered once daily for 30 days to 9-month old MPS IIIB. The breeding pairs of MPS IIIB mice were obtained from Jackson Laboratory (JAX stock #003827) and a MPS IIIB mouse colony was established. In MPS IIIB or Sanfillipo syndrome mice, both alleles of alpha-N-acetylglucosaminidase (NAGLU) are disrupted, knocked out (KO), by insertion of a neomycin resistance gene via homologous recombination. Gografe S I; Garbuzova-Davis S; Willing A E; Haas K; Chamizo W; Sanberg P R. Mouse model of Sanfilippo syndrome type B: relation of phenotypic features to background strain Comp Med 53(6):622-32. Homozygous (KO) mutant mice are negative for NAGLU enzyme activity, which metabolizes heparan sulfate (HS) in the lysosome, which results in a buildup of incompletely processed HS in the lysosome and in and around the cell. The heterozygous (Het) mice have only one allele of the NAGLU gene disrupted and can fully metabolize HS.

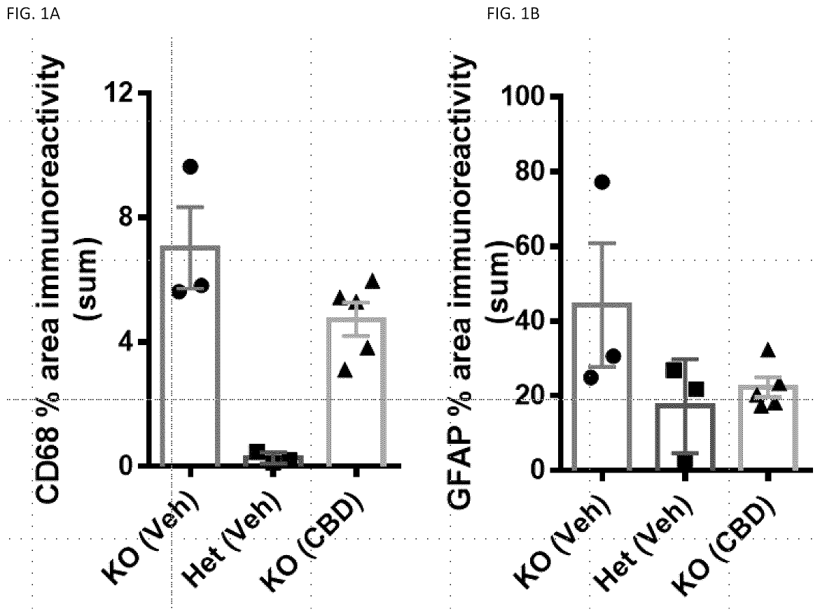

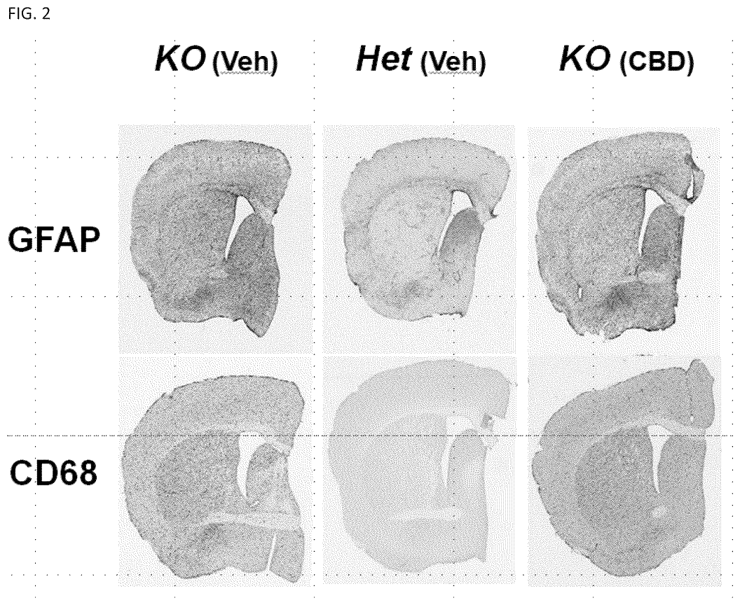

Specifically, the KO mice (n=5) were dosed once daily by oral gavage for 30 days with 100 mg/kg CBD in sesame oil, or KO mice (n=3) or Het mice (n=3) were dosed with sesame oil vehicle (Veh) on the same schedule. Results on certain biomarkers in the brain are shown in FIGS. 1A, 1B and 2. FIG. 1A and FIG. 1B show bar graphs of the percentage staining of Cluster of Differentiation 68 (CD68) and glial fibrillary acidic protein (GFAP), respectively, in serial section (one every twelve sections of 40 um thick) along the rostrocaudal axis of the brain after 30 days. One in every 12 brain sections (40 um each) were immunostained using specific antibodies for CD68, and GFAP.

Quantification of brain slices was performed using Image Pro Premiere software.

Specifically, the percentage area of immunoreactivity for (FIG. 1A) CD68 and (FIG. 1B) GFAP was measured in brains of MPS IIIB mice (KO) given CBD or sesame oil (Veh), and the heterozygote (Het) control mice given sesame oil (Veh). After 30 days, animals were sacrificed and brain slices were taken along the rostro caudal axis in a serial section. Quantification was performed using Image Pro Premiere software.

FIG. 1A shows there was a 30% decrease in the immunoreactivity of CD68 in the MPS IIIB KO mice treated with CBD, compared to the KO mice receiving vehicle (Veh).

FIG. 1B shows there was a 50% decrease in the immunoreactivity of GFAP in the MPS IIIB KO mice treated with CBD, compared to the KO mice receiving vehicle (Veh).

FIG. 2 shows representative brain slices showing the impact of CBD treatment on cellular neuroinflammation. Shown are representative brain slices taken along the rostro caudal axis in a serial section and immunostained for GFAP or CD68 proteins from MPS IIIB mice (KO) given CBD, or sesame oil (Veh), and the heterozygote control mice (Het) given sesame oil. As shown, CBD treatment significantly decreases both microglia and astrocyte cell neuroinflammation and activation, as indicated by the reduction in CD68 and GFAP associated with microglial and astrocyte cells activation, respectively.

### Example 2. Effect of CBD in Behavioral Mouse Model of MPS III B

An oral formulation of 98% plus purity CBD or the formulation without CBD (vehicle control) is administered once daily to mice suffering from the lysosomal storage disease MPS IIIB (KO) described in Example 1, and to control wild type mice (WT). CBD or vehicle is administered daily by oral gavage. CBD is administered at doses of 100 mg/kg/day, 33 mg/kg/day or 10 mg/kg/day for 9 months to KO and WT mice (each n=24). At baseline, and after 3, 6, and 9 months, the mice are given behavioral tests to measure hyperactive and social interaction, and cognitive tests to measure spatial memory and learning deficits. After 6 months of CBD dosing, there is expected to be less hyperactivity, more social interaction, less loss of spatial memory, and less learning deficit in the MPS IIIB KO mice treated with CBD than the MPS IIIB mice treated with the oral formulation that does not contain CBD (vehicle control).

### Example 3. Effect of CBD on Inflammatory Signaling Molecules in Mouse Model of MPS IIIB

An oral formulation of 98% plus purity CBD, or sesame oil vehicle (Veh) is administered by oral gavage once daily to mice suffering from the lysosomal storage disease MPS IIIB (KO), as described in Example 1. CBD is dosed at 100 mg/kg/day, 33 mg/kg/day or 10 mg/kg/day. At baseline and after 6 and 9 months, plasma and CSF of MPS IIIB KO mice given CBD or vehicle are measured for inflammatory signaling molecules. After 6 months of CBD dosing, there are is expected to be less inflammatory signaling molecules in the plasma and CSF of MPS IIIB mice treated with CBD than the control mice treated with the oral formulation that does not contain CBD (vehicle control). In particular, less of one or more of IL-1alpha, IL1beta, IL-2, IL-4, IL-6, IL-8, 11-10, IL-17A, IL-23, INF-gamma, MCP-1, caspase-1, caspase-3, TNF-alpha, and TGF-beta-alpha is expected.

### Example 4. Identification of CBD Receptors Via Affinity Chromatography

Historically, a receptor for a small molecule like CBD is discovered by using CBD to “fish” for the receptor by affinity-based methods. One method for identifying CBD receptors is to use a CBD-affinity chromatography where CBD is bound to a bead and packed into a column, then cell or brain lysate is poured into the column. Receptors that bind CBD are retained on the column and can be eluted and characterized.

### Example 5. Identification of CBD Receptors Via Proteomic Target Identification

Another method is to use Proteomic Target Identification to identify CBD receptors. This method relies on the observation that receptors are more stable when bound to a small molecule, which makes them less susceptible to proteolysis. Enzymatic or chemical lysates are compared in the presence and absence of a small molecule. Receptors that bind to the small molecule are protected from proteolysis relative to the control sample. A similar method relies on the irreversible oxidation of methionine residues by hydrogen peroxide to report on the thermodynamic stability of a receptor's structure during chemical denaturation techniques.

### Example 6. Identification of CBD Receptors Via Covalent Chemical Warheads

It is possible CBD does not bind the receptor in lysates, but may require the context of a cell for binding to the receptor. A method that relies on CBD binding covalently to the receptor on the surface of a cell by derivitization of CBD to a “chemical warhead” that forms an irreversible covalent bond to a receptor that binds CBD. For example, one or both of the hydroxyl moieties on CBD may be directly derivitized with a chemical warhead, or may be derivitized with an amino acid by any method known in the art to obtain a derivitizable amino group, such as a valine, which may in turn be derivatized with a succinimide to obtain a derivitizable carboylate moiety, for example, as described in Taskar et al., 2019 Analog Derivitization of Cannbidiol for improved ocular permeation, J Ocular Pharmacol and Therapeutics, 35, 5, DOI: 10.1089/jop.2018.0141. For example, exposure to UV-light activates the chemical warhead to form a covalent bond. For example, Martin-Gago et al. 2017, Cell Chemical Biology, 24, 589-597 describe use of photo-activatable tetrazoles or Woodwards reagent K for preparing covalent probes for selective labeling of glutamic acid residues inside protein binding pockets.

### REFERENCES CITED

1. Atri, Alireza. “The Alzheimer's Disease Clinical Spectrum: Diagnosis and Management.” The Medical Clinics of North America 103, no. 2 (March 2019): 263-93.
2. Brown, Robert H., and Ammar Al-Chalabi. “Amyotrophic Lateral Sclerosis.” The New England Journal of Medicine 377, no. 2 (Jul. 13, 2017): 162-72.
3. Castillo J. Evolving paradigms for neuroprotection: molecular identification of ischemic penumbra. Cerebrovasc Dis. 2006; 21(suppl 2):71-79.
4. Gao, Hui-Ming, Janwei Jiang, Belinda Wilson, Wanqin Zhang, Jau-Shyong Hong, and Bin Liu. “Microglial Activation-Mediated Delayed and Progressive Degeneration of Rat Nigral Dopaminergic Neurons: Relevance to Parkinson's Disease.” Journal of Neurochemistry 81, no. 6 (June 2002): 1285-97.
5. Gografe S I; Garbuzova-Davis S; Willing A E; Haas K; Chamizo W; Sanberg P R. Mouse model of Sanfilippo syndrome type B: relation of phenotypic features to background strain Comp Med 53(6):622-32.
6. Griffin, W. Sue T., Ling Liu, Yuekui Li, Robert E. Mrak, and Steven W. Barger. “Interleukin-1 Mediates Alzheimer and Lewy Body Pathologies.” Journal of Neuroinflammation 3 (Mar. 16, 2006): 5.
7. Jurga, Agnieszka M., Martyna Paleczna, Justyna Kadluczka, and Katarzyna Z. Kuter. “Beyond the GFAP-Astrocyte Protein Markers in the Brain.” Biomolecules 11, no. 9 (Sep. 14, 2021): 1361
8. Martin-Gago et al. 2017 Exposure to UV-light activates the chemical warhead to form a covalent bond. Cell Chemical Biology, 24, 589-597.
9. Millar, Sophie A., Nicole L. Stone, Andrew S. Yates, and Saoirse E. O'Sullivan. “A Systematic Review on the Pharmacokinetics of Cannabidiol in Humans.” Frontiers in Pharmacology 9 (2018).
10. Nelson, Peter T., Lorinda A. Soma, and Ehud Lavi. “Microglia in Diseases of the Central Nervous System.” Annals of Medicine 34, no. 7-8 (2002): 491-500.
11. Polgreen, Lynda E., Alicia Kunin-Batson, Kyle Rudser, Richard K. Vehe, Jeanine J. Utz, Chester B. Whitley, and Patricia Dickson. “Pilot Study of the Safety and Effect of Adalimumab on Pain, Physical Function, and Musculoskeletal Disease in Mucopolysaccharidosis Types I and II.” Molecular Genetics and Metabolism Reports 10 (Jan. 15, 2017): 75-80.
12. Taskar et al., 2019 Analog Derivitization of Cannbidiol for improved ocular permeation, J Ocular Pharmacol and Therapeutics, 35, 5.
13. Waller, Rachel, Lynne Baxter, Daniel J. Fillingham, Santiago Coelho, Jose M. Pozo, Meghdoot Mozumder, Alejandro F. Frangi, Paul G. Ince, Julie E. Simpson, and J. Robin Highley. “Iba-1−/CD68+ Microglia Are a Prominent Feature of Age-Associated Deep Subcortical White Matter Lesions.” PLoS ONE 14, no. 1 (Jan. 25, 2019): e0210888.

1. A method for preventing or reducing acute or chronic neuroinflammation or neurodegeneration, and/or preventing or treating a disease or disorder associated with acute or chronic neuroinflammation in a subject in need thereof, comprising administering to the subject a composition comprising a therapeutically effective amount of a cannabinoid or functional equivalent and a pharmaceutically acceptable carrier.

2.-3. (canceled)

4. The method according to claim 1, wherein the cannabinoid or functional equivalent is cannabidiol (CBD).

5. The method according to claim 1, wherein the CBD is a highly purified extract of Cannabis which comprises at least 98% (w/w) CBD.

6. The method according to claim 5, wherein the Cannabis is a Cannabis sativa.

7. The method according to claim 1, wherein the CBD is a highly purified or synthetic CBD which comprises at least 95% (w/w) CBD and comprises no more than 2% THC and a pharmaceutically acceptable carrier.

8. The method according to claim 1, wherein the CBD is a highly purified or synthetic CBD which comprises at least 98% (w/w) CBD, and a pharmaceutically acceptable carrier.

9. The method according to claim 1, wherein the CBD is a highly purified or synthetic CBD which comprises at least 99% (w/w) CBD, and a pharmaceutically acceptable carrier.

10. The method according to claim 1, wherein the composition is administered by an oral, sub-lingual, buccal, sub-cutaneous, intramuscular, intraperitoneal, intracerebrovascular injection, intrarectal, enema or suppository, intrathecal, intravenous, intra-nasal, intra-lesion, topical, transdermal, transmucosal, or inhalation route.

11. The method according to claim 1, wherein the composition is effective to reduce severity, duration, and/or delays onset of neuroinflammation in the subject.

12. The method according to claim 1, wherein the disease, disorder, or condition is selected from the group consisting of Lysosomal Storage Disease, Alzheimer's disease, Parkinson's disease, Amyotrophic Lateral Sclerosis, Lewy body dementia, Multiple Sclerosis, stroke, spinal cord injury, subacute combined degeneration of spinal cord, traumatic brain injury, CNS vasculitis, depression, schizophrenia, infection of the brain, infection of the central nervous system, and brain tumors.

13. The method according to claim 12, wherein the disease, disorder, or condition is mucopolysaccharidosis III.

14. The method according to claim 13, wherein the disease, disorder, or condition is mucopolysaccharidosis IIIA or IIIB.

15. The method according to claim 1, wherein the reduction of severity or duration or both delayed onset of neuroinflammation in the subject comprises reduction of microglia activation or reduction of astrocyte activation or both, or reduction of the secretion of inflammatory signaling molecules.

16. The method according to claim 15, wherein reduction of microglia cell activation is evidenced by a decrease in the expression of CD68.

17. The method according to claim 15, wherein reduction of astrocyte cell activation is evidenced a decrease in the expression of GFAP.

18. The method according to claim 1, wherein the reduction of severity or duration or both delayed onset of neuroinflammation in the subject comprises reduction of the secretion of inflammatory signaling molecules.

19. The method according to claim 18, wherein the reduction of the secretion of inflammatory signaling molecules comprises a reduction in a signaling molecule selected from the group consisting of IL-1-alpha, IL-1-beta, IL-2, IL-4, IL-6, IL-8, 11-10, IL-17A, IL-23, INF-gamma, CCL2 (MCP-1), CXCL10 IP-10), CXCL1 (KC), MCP-1, CCL3 (MIP-1α), MIP-2, CCL5 (RANTES), caspase-1, caspase-3, TNF-alpha, and TGF-beta.

20. The method according to claim 1, wherein the effective amount CBD is within a range of from about 5 to about 100 mg/kg/day, about 10 to about 50 mg/kg/day, or about 20 to about 30 mg/kg/day.

21.-22. (canceled)

23. A composition for reducing severity or duration of symptoms of a disease or disorder associated with neuroinflammation in a subject in need thereof, the composition comprising a therapeutically effective amount of CBD and a pharmaceutically acceptable carrier.

24. (canceled)
